(19) United States
(12) Patent Application Publication
(10) Pub. No.: US 2017/0333380 A1
(43) Pub. Date: Nov. 23, 2017

(54) ANIMAL FEED ADDITIVE AND METHOD FOR USING SAME

(71) Applicants: Kwan-Sik YUN, Bucheon-si (KR); KIMIN INC., Seoul (KR)

(72) Inventor: Kwan-Sik YUN, Bucheon-si (KR)

(21) Appl. No.: 15/596,536

(22) Filed: May 16, 2017

(30) Foreign Application Priority Data
May 17, 2016 (KR) 10-2016-0060008

(51) Int. Cl. A61K 31/23; A23K 50/30; A23K 20/20; A23K 50/75; A23K 20/163

(52) U.S. Cl. CPC A61K 31/23; A23K 50/75; A23K 20/163; A23K 20/20; A23K 50/30

(57) ABSTRACT

Provided is an animal feed additive including: 20 to 50 wt % of a salt of lactic acid and stearic acid ester; and 50 to 80 wt % of one kind of salt selected from the group consisting of a salt of lactic acid and palmitic acid ester, a salt of lactic acid and lauric acid ester, and mixtures thereof. The lactic acid is a monomer or a dimer. The salt is a sodium salt, a calcium salt, or a potassium salt.

### BACKGROUND

The present disclosure relates to an animal feed additive which, by increasing the utilization efficiency in the body of fat present in the animal feed, decreases the amount of fat required in the feed and improves productivity, and to a method for using the same.

Feed refers to material that supplies organic or inorganic nutrients necessary for livestock survival and for producing milk, meat, eggs, and furs and the like. Feed is produced by uniformly mixing not only nutrients, such as energy, protein, vitamins, and minerals, required by various livestock, but also growth promotants and disease prevention agents.

Feed not only supplies, via consumption by livestock, nutrients necessary for livestock survival and the production of animal products, but also performs various roles, such as enhancing immune function, improving the quality of the animal products, and improving the environment of stables.

In particular, increased livestock productivity is achieved by improving the environment of stables or by improving feed efficiency. Research is being carried out on various methods of improving feed efficiency, such as adding new compositions to existing compositions, modifying mixture ratios, or changing feeding methods.

Most animals are capable of accumulating fat nearly without limit, and thus are able to store excess energy in the form of fat. Such fat is the source of essential fatty acids, is present also in the protoplasm of cells as a component of the body, and being present in brain tissue, nerve tissue, the liver and the like, performs important physiological activities. Fat protects important organs in the body from external impact, performs the role of preventing body temperature decline, and also performs the role in the body of a buffer protecting the host from disease. Moreover, when the amount of chemically or physiologically harmful substances in the blood exceeds a safe level, fat assists in protecting important organs during the removal of problematic harmful substances by storing the harmful substances in new fat tissue, thereby diluting the harmful substances in the body or maintaining a minimum equilibrium state.

Such fat is one of the essential nutrients for livestock, and fat present in feed includes, in addition to extracted fat, such as soybean oil or tallow, intact fat present in grain material. Recently, due to huge increases in biofuel production and the consumption in China of grain, the price of feed ingredients has increased sharply such that the burden on common farmers of the cost of feed is continually growing. Thus, if the utilization efficiency of fat—higher in energy than other nutrients and the most expensive energy source per unit weight—in the bodies of livestock is enhanced, not only can livestock productivity be improved, but an opportunity may be presented for reducing production cost by reducing the raw material cost of feed.

Accordingly, various research is being carried out on increasing the utilization of fat, which is an energy resource in feed, and for this end, mostly protected fats, emulsifiers, and the like in feed are being used.

Most research on adding emulsifiers into feed to improve the utilization efficiency of fat in livestock uses lecithin, which is a water-in-oil type (lipophilic) emulsifier. Meanwhile, methods have been proposed for feeding animals by using an emulsifier to artificially emulsify fat in feed. However, since most of the fat broken down by lipase in the bodies of animals is absorbed in mucosa in the small intestine, it is difficult for water-in-oil type (lipophilic) emulsifiers such as lecithin to form a ball of fat in the digestive tract. Moreover, the feeding of fat emulsified by an emulsifier is limited in that it is impossible to achieve improvement in the utilization efficiency of other types of fat present in the feed.

As described, in addition to being used in feed to improve the utilization of fat, emulsifiers are also used, by making use of the property thereof of emulsifying oil and water, for reducing the surface tension between two materials which, among those added to the feed, do not easily mix with each other, to thereby uniformly disperse the materials.

Meanwhile, sodium stearoyl-2-lactylate (hereinafter referred to as SSL) is produced by esterification of lactic acid and stearic acid, and neutralized by food grade sodium carbonate or concentrated sodium hydroxide and the like. SSL has an HLB value near 20 and is thus hydrophilic. Moreover, SSL is slightly hygroscopic, is a useful oil-in-water type (hydrophilic) emulsifier, and can also function as a wetting agent.

Such SSL is variously applied in animal feed and the like. For example, in U.S. Patent Publication No. 2008-0063775, SSL is used as a lubricating agent in dog chew, while U.S. Pat. No. 4,351,735, Chinese Patent Publication No. 001883294, International Patent Publication No. WO 98/09538, and U.S. Patent Publication No. 2008-0160133 disclose the use of SSL as an emulsifier in feed or food and the like which is fed to animals.

In another example, British Patent Publication No. 1581744 indicates that SSL may be used in the feed of salmon and the like to enhance the absorbability of vitamins, vitamin precursors, and pigments, U.S. Patent Publication No. 2009-0041888 indicates that the use of an emulsifier such as SSL and the like in fish feed can prevent the phenomenon, which occurs in the digestive tract of fish, of stratification into undigested food particles, digested food particles, and lipid, and thereby increase absorption of nutrients, and International Patent Publication No. 1996-013175 indicates that negative effects caused by coccidiosis may be reduced by using a mixture of SSL and cresols, thymol, capsaicin, tannin, etc.

With regard to novel uses of SSL, the present inventors have received Korean Patent No. 10-0960531, which relates to the use of SSL as a bile salt supplement which enhances the absorption efficiency of fat in the body by emulsifying fat in the body into smaller particles when fed to animals.

### PRIOR ART DOCUMENTS

### Patent Documents

(Patent Document 1) U.S. Patent Publication No. 2008-0063775: HIGH AMYLOSE DOG CHEW FORMULATION

(Patent Document 2) U.S. Pat. No. 4,351,735: MINERAL ENRICHMENT COMPOSITION AND METHOD OF PREPARING SAME

(Patent Document 3) Chinese Patent Publication No. 001883294: FORMULATION OF WATER SOLUBLE MICRO-CAPSULE FAT POWDER FEED AND MANUFACTURING PROCESS THEREOF

(Patent Document 4) International Patent Publication No. WO 1998-09538: ANIMAL FEEDSTUFFS

(Patent Document 5) U.S. Patent Publication No. 2008-0160133: LOW FAT, WHEY-BASED CREAM CHEESE PRODUCT WITH CARBOHYDRATE-BASED TEXTURIZING SYSTEM AND METHODS OF MANUFACTURE

(Patent Document 6) British Patent Publication No. 1581744: EDIBLE COMPOSITION

(Patent Document 7) U.S. Patent Publication No. 2009-0041888: USE OF FORMULATED DIETS PROVIDING IMPROVED DIGESTION IN FISH

(Patent Document 8) International Patent Publication No. WO 1996-013175: POULTRY FEED ADDITIVE COMPOSITION

### SUMMARY

The present disclosure provides an animal feed additive capable of enhancing livestock productivity by increasing the utilization efficiency in the body of fat present in the feed.

The present disclosure also provides an animal bile salt supplement capable of assisting bile salt present in the body of animal in increasing the absorption efficiency in the body of fat present in the feed.

The present disclosure further provides a method for reducing the amount of fat required in feed or improving productivity by feeding animals with the animal feed additive or animal bile salt supplement to enhance the absorption efficiency of fat present in the feed.

In accordance with an exemplary embodiment of the present invention, an animal feed additive includes: 20 to 50 wt % of a salt of lactic acid and stearic acid ester; and 50 to 80 wt % of one kind of salt selected from the group consisting of a salt of lactic acid and palmitic acid ester, a salt of lactic acid and lauric acid ester, and mixtures thereof

In accordance with another exemplary embodiment of the present invention, an animal bile salt supplement includes: 20 to 50 wt % of a salt of lactic acid and stearic acid ester; and 50 to 80 wt % of one kind of salt selected from the group consisting of a salt of lactic acid and palmitic acid ester, a salt of lactic acid and lauric acid ester, and mixtures thereof

In accordance with another exemplary embodiment of the present invention, a method for reducing the content of fat required in feed by enhancing the absorption ratio of fat present in the feed includes feeding animals with the animal feed additive.

In accordance with another exemplary embodiment of the present invention, a method for reducing the content of fat required in feed by enhancing the absorption ratio of fat present in the feed includes feeding animals with the animal bile salt supplement.

In accordance with another exemplary embodiment of the present invention, a method for enhancing productivity includes feeding animals with the animal feed additive.

In accordance with another exemplary embodiment of the present invention, a method for enhancing productivity includes feeding animals with the animal bile salt supplement.

In accordance with still another exemplary embodiment of the present invention, an animal feed composition includes the animal feed additive and a formulated feed.

### DETAILED DESCRIPTION OF EMBODIMENTS

Hereinafter, the present invention is described in greater detail.

An animal feed additive or animal bile salt supplement of the present invention includes 20 to 50 wt % of a salt of lactic acid and stearic acid ester; and 50 to 80 wt % of one kind of salt selected from the group consisting of a salt of lactic acid and palmitic acid ester, a salt of lactic acid and lauric acid ester, and mixtures thereof

Typically, SSL includes 70 to 90 wt % of a sodium salt of lactic acid and stearic acid ester and 10 to 30 wt % of a sodium salt of lactic acid and palmitic acid ester, and has a melting point of 50 to 55° C.

In the present invention, the content of the salt of lactic acid and palmitic acid ester in SSL is increased to 50 to 80 wt %, desirably 60 to 80 wt %, or 50 to 80 wt %, desirably 60 to 80 wt %, of the salt of lactic acid and lauric acid ester is added to lower the melting point of SSL to 30 to 43° C., and consequently, the solubility in an animal body is improved. Here, when the content of the salt of lactic acid and palmitic acid ester and the content of the salt of lactic acid and lauric acid ester are below the above ranges, reducing the melting point is difficult. Conversely, when the content of the salt of lactic acid and palmitic acid ester and the content of the salt of lactic acid and lauric acid ester exceed the above ranges, it is difficult to achieve the effect of enhancing the utilization efficiency of fat in the body.

The lactic acid may be a monomer or a dimer, and the salt is a sodium salt, calcium salt, or potassium salt.

Fat contained in a feed composition cannot be absorbed as-is, but can be absorbed in the intestines after being broken down by lipase. Here, in order for lyase activity to be efficient, the surface area of the fat must be maximized through the activity in the intestines of bile salt, which is a bioemulsifier, to form as small a fat droplet as possible. The smaller the size of the fat droplet, the larger the relative surface area is, and thus the faster and more compete is the breakdown of the fat. Moreover, in order to be absorbed, broken down fatty acids form small balls of fat, called micelles, before being absorbed into small intestine cells. Here, absorption efficiency increases as the size of the micelles decreases.

In the case of young livestock, feed having a relatively high content of fat as an energy source is fed, even though villi in the intestines are not sufficiently developed, and thus the ability to produce and utilize enzymes and bile salt necessary for digestion is extremely limited. Accordingly, a reduction in emulsifying ability due to insufficient bile salt lowers the activity of lipase, thereby reducing the utilization of fat. Not only that, even in the case of adult livestock, in which the secretion of bile salt and lipase is sufficient, the digestibility of fat is not high. This is because, extracted fat, such as soybean oil or tallow, added to the feed has a physical structure that is more easily digested or absorbed in the body than intact fat contained in grain ingredients, whereas, intact fat contained in grain ingredients is surrounded by cell membrane, and thus is more limited than extracted fat, such as soybean oil or tallow, in terms of being emulsified or digested.

The animal feed additive or animal bile salt supplement according to the present invention not only assists the bile salt present in the bodies of animals by forming fat in the feed into small balls of fat to increase the surface area of the fat, but also increases the absorption efficiency of both added extracted fat, such as soybean oil and tallow, and intact fact, contained in grain ingredients, present in the feed, by minimizing the size of micelles prior to absorption.

Thus, the animal feed additive or animal bile salt supplement according to the present invention may reduce the amount of fat used in the feed by enhancing the absorption ratio of the fat present in the feed when the feed is fed to animals.

Moreover, the animal feed additive or animal bile salt supplement according to the present invention may improve livestock productivity by enhancing the utilization efficiency of fat present in the feed when the feed is fed to animals.

In the animal feed additive or animal bile salt supplement of the present invention, when considering the environment inside the bodies of animals, in which metabolism occurs mostly through moisture, in particular that inside the small intestine, in which the absorption of fat occurs, it is desirable for the salt of lactic acid and stearic acid ester, the salt of lactic acid and palmitic acid ester, and the salt of lactic acid and lauric acid ester to be in a non-dissociated state.

SSL used in typical feed is mostly used as an emulsifier. SSL added as an emulsifier during feed production is ionically dissociated in an aqueous solution during emulsification, and an anion part exhibits surface activity. Thus, even when an emulsifier, which has already been dissociated when added to feed as an emulsifier, is fed to an animal, it is difficult for the emulsifier to exhibit such surface activity inside the body of the animal, due to the fact that the emulsifier has already been ionically dissociated.

Thus, the animal feed additive or animal bile salt supplement of the present invention, rather than being fed in an emulsified state mixed with oil-based components and water-based components, is fed in a non-dissociated state, and thus may exhibit the surface activity by being ionically dissociated inside the body of an animal.

The animal feed additive or animal bile salt supplement of the present invention may be added, in the form of an additive, to a formulated feed.

An animal feed composition of the present invention includes 0.01 to 5.0 wt %, desirably 0.01 to 1.0 wt %, of the animal feed additive or animal bile salt supplement with respect to the total weight of the animal feed composition. When the content of the animal feed additive or animal bile salt supplement is below the above range, it is difficult to expect an effect of using the same, whereas, when the content exceeds the above range, the nutritional status of livestock being raised may become unbalanced.

The animal feed composition may be formed by adding the animal feed additive or animal bile salt supplement to an arbitrary formulated feed disclosed in the field or commercially available. The formulated feed for livestock varies in composition and production method according to the type of livestock being fed, and thus in the present invention, the composition and production method are not particularly limited. Here, the livestock is pig, chicken, duck, quail, goose, pheasant, turkey, cattle, milk cow, horse, donkey, sheep, goat, dog, cat, rabbit, and various types of farmed fish and shrimp.

In addition, the feed composition according to the present invention may include, when needed, various antibiotics, probiotics, enzyme preparations, organic acids, flavoring agents, sweeteners, antioxidants, and other functional substances to improve the health of animals or enhance productivity and achieve a positive effect for producing high-quality animal products.

The feed composition according to the present invention may be fed to livestock according to the purpose of raising the livestock, for a typical period of time it takes for the livestock to reach an appropriate weight.

When the animal feed additive according to the present invention was used to feed animals, it was shown that productivity tended to improve even when the feed energy content was lowered by reducing the level of added fat by 0.5% compared to a control group. Even when the amount of added fat was reduced by up to 1% compared to the control group, there was no effect on productivity.

Hereinafter, exemplary embodiments and experimental examples are provided. However, the examples below are merely exemplary examples of the present invention, and the present invention is not limited to such examples.

### Examples and Comparative Examples

Additives having the compositions in Table 1 below were used in the following experiments.

TABLE 1

| | | | | | Comparative | Comparative | Comparative | Comparative |
|---|---|---|---|---|---|---|---|---|
| Item (wt %) | Example 1 | Example 2 | Example 3 | Example 4 | Example 1 | Example 2 | Example 3 | Example 4 |
| Sodium salt of | 20 | 40 | 20 | 40 | 60 | 80 | 60 | 80 |
| lactic acid and | | | | | | | | |
| stearic acid ester | | | | | | | | |
| Sodium salt of | 80 | 60 | — | — | 40 | 20 | — | — |
| lactic acid and | | | | | | | | |
| palmitic acid ester | | | | | | | | |
| Sodium salt of | — | — | 80 | 60 | — | — | 40 | 20 |
| lactic acid and | | | | | | | | |
| lauric acid ester | | | | | | | | |

### Experimental Example 1

### Melting Point Measurement Using DSC

Using a differential scanning calorimeter (DSC), the melting points of the additives of Examples 1 and 2 and Comparative Examples 1 and 2 were measured using an EXO DOWN technique, and the results thereof displayed in Table 2.

TABLE 2

| | | | | | Comparative | Comparative | Comparative | Comparative |
|---|---|---|---|---|---|---|---|---|
| Item | Example 1 | Example 2 | Example 3 | Example 4 | Example 1 | Example 2 | Example 3 | Example 4 |
| Melting | 40.32 | 42.91 | 32.23 | 36.95 | 48.17 | 51.74 | 44.76 | 49.12 |
| point (° C.) | | | | | | | | |

### Experimental Example 2

### Test Of Effect on Mouse Growth

Feed for laboratory animals, purchased from Shincheon Feed Inc., was ground and then made into molded feed of a predetermined size by adding various additives.

Male ICR mice, 4 weeks old and weighing 11.5 to 13.5 g, were purchased from the Seoul National University Hospital Laboratory Animal Center, divided into five groups of 12 mice each, maintained at a temperature of 23±2° C., and fed various types of special feed. Here, medication of the animals in the respective groups was carried out as follows (% indicates wt %): in order to dissociate the salts, the salts were washed using a separatory funnel and then used after being dried.
Control Group: feed +5% of soybean oil
Experimental Group 1: feed +5% of soybean oil +0.1% of feed additive of Example 1 in non-dissociated state
Experimental Group 2: feed +5% of soybean oil +0.1% of feed additive of Example 2 in non-dissociated state
Experimental Group 3: feed +5% of soybean oil +0.1% of feed additive of Example 3 in non-dissociated state
Experimental Group 4: feed +5% of soybean oil +0.1% of feed additive of Example 4 in non-dissociated state
Comparative Group 1: feed +5% of soybean oil +0.1% of feed additive of Example 4 in dissociated state
Comparative Group 2: feed +5% of soybean oil +0.1% of feed additive of Comparative Example 1 in non-dissociated state
Comparative Group 3: feed +5% of soybean oil +0.1% of feed additive of Comparative Example 2 in non-dissociated state
Comparative Group 4: feed +5% of soybean oil +0.1% of feed additive of Comparative Example 3 in non-dissociated state
Comparative Group 5: feed +5% of soybean oil +0.1% of feed additive of Comparative Example 4 in non-dissociated state

### (1) Change in Mouse Weight

The weights for the respective groups were measured prior to feeding and after 6, 16, and 25 days of feeding. The amount of feed intake was also measured.

TABLE 3

| Weight (g) | 0 days | 6 days | 16 days | 25 days |
|---|---|---|---|---|
| Control Group | 12.9 ± 1.43 | 21.6 ± 1.55 | 29.6 ± 1.39 | 35.8 ± 2.78 |
| Experimental | 12.9 ± 1.61 | 22.6 ± 1.95 | 31.3 ± 1.09 | 36.7 ± 1.73 |
| Group 1 | | | | |
| Experimental | 13.0 ± 1.57 | 21.6 ± 1.90 | 30.3 ± 1.57 | 36.4 ± 1.40 |
| Group 2 | | | | |
| Experimental | 13.1 ± 1.26 | 22.2 ± 1.80 | 30.9 ± 1.91 | 36.2 ± 1.85 |
| Group 3 | | | | |
| Experimental | 12.8 ± 1.42 | 21.4 ± 1.75 | 30.1 ± 1.75 | 36.0 ± 1.27 |
| Group 4 | | | | |
| Comparative | 13.0 ± 1.21 | 21.2 ± 1.31 | 29.3 ± 1.34 | 35.2 ± 2.03 |
| Group 1 | | | | |
| Comparative | 12.5 ± 1.50 | 21.1 ± 1.79 | 29.5 ± 3.93 | 35.7 ± 1.82 |
| Group 2 | | | | |
| Comparative | 13.4 ± 1.54 | 21.7 ± 1.98 | 29.2 ± 2.01 | 35.3 ± 2.46 |
| Group 3 | | | | |
| Comparative | 13.1 ± 1.74 | 21.9 ± 1.51 | 29.7 ± 1.84 | 35.9 ± 1.53 |
| Group 4 | | | | |
| Comparative | 13.2 ± 1.20 | 21.5 ± 1.39 | 29.4 ± 2.33 | 35.6 ± 1.92 |
| Group 5 | | | | |

Referring to Table 3 above, it can be seen that weight gain was greatest for the experimental groups that consumed the feed additives according to the present invention. (2) Mouse small intestine propulsion measurement

After the final weight measurement, four mice from each group were orally administered with 0.2 ml (per mouse) of a BaSO₄ suspension (BaSO₄:H₂O=1:1), and 30 minutes later, the mice underwent cervical dislocation and were opened to measure the distance traveled by BaSO₄ in the small intestine. Small intestine propulsion was determined using the ratio (%) of distance traveled to total small intestine length.

TABLE 4

| | | Exper- | | | | | | | | |
|---|---|---|---|---|---|---|---|---|---|---|
| | Control | imental | Experimental | Experimental | Experimental | Comparative | Comparative | Comparative | Comparative | Comparative |
| Treatment | Group | Group 1 | Group 2 | Group 3 | Group 4 | Group 1 | Group 2 | Group 3 | Group 4 | Group 5 |
| Propulsion | 41.1 ± 4.4 | 33.6 ± 4.6 | 36.4 ± 4.0 | 35.6 ± 3.7 | 37.4 ± 2.8 | 40.1 ± 2.6 | 40.3 ± 3.4 | 42.1 ± 5.0 | 39.7 ± 2.4 | 41.7 ± 3.1 |
| (%) | | | | | | | | | | |

The small intestine propulsion experiment is similar to tests of diarrhea frequency, and lower values thereof indicate lower frequencies of diarrhea.

Referring to Table 4 above, it can be seen that the small intestine propulsion of the Control Group and the Comparative Groups is high, and thus the diarrhea frequencies of animals is high following feed intake. In contrast, the values are low for the Experimental Groups, which have consumed the feed additives according to the present invention, and through these results, it can be seen that there is potential for preventing dietary diarrhea by improving the absorption ratio of fat through the use of SSLs having reduced melting temperatures.

### (3) Fecal Fat Content Analysis

Mice from each of the groups were placed under a fasting condition for one evening in a metabolic cage, and after collecting the feces, the fat content was measured using a Soxhlet extraction method.

TABLE 5

| | After 10 days of | After 25 days of |
|---|---|---|
| Treatment | feed intake (%) | feed intake (%) |
| Control Group | 6.60 | 4.42 |
| Experimental Group 1 | 4.30 | 3.52 |
| Experimental Group 2 | 4.92 | 3.66 |
| Experimental Group 3 | 5.11 | 3.81 |
| Experimental Group 4 | 5.45 | 3.87 |
| Comparative Group 1 | 6.06 | 4.07 |
| Comparative Group 2 | 6.16 | 4.16 |
| Comparative Group 3 | 6.39 | 4.21 |
| Comparative Group 4 | 5.96 | 4.28 |
| Comparative Group 5 | 6.46 | 4.39 |

Referring to Table 5, after 10 days of feed intake, the fat content was lowest in the feces of the Experimental Groups, and since this tendency was also observed in the values obtained after 25 days of feeding, it can be seen that the absorption ratio in the body is high.

### Experimental Example 3

### Test Of Effect On Growing Pig Growth

Sentinel animals were each raised in a growth facility, equipped with mechanical ventilation equipment, in TS Corporation's Ansung Test Farm. A pigsty was designed with slat flooring, and a single hole wet feeder was installed to allow free intake of feed and water.

For testing, a total of 120 castrated and female growing pigs (Landrace x Yorkshire×Duroc) weighing an average of 29.97 kg were used, and each treatment group was tested three times over a total of four weeks.

The sentinel animals were allocated, four females and four castrated pigs per pen, on the basis of baseline weight and sex, and three pens each were allocated per treatment group in a completely randomized fashion (5×3 randomized complete block design). The sentinel animals were weighed at the start and end of the test, and the amount of feed intake was measured each week.

A treatment group designed for a net energy of 2,320 kcal/kg was set as the Control Group. The composition ratios and design components of test feeds used in the experiment are displayed in Tables 6 and 7 below. Chemical components in the test feed were analyzed using the method of AOAC (1990).

In Experimental Groups 1 and 3, 0.5% of animal fat was excluded (replaced with corn) and 0.05% was added of the feed additives of Examples 1 and 3, respectively. In Experimental Groups 2 and 4, using the Control Group as the baseline, 1.0% of animal fat was excluded (replaced with corn) and 0.05% was added of the feed additives of Examples 1 and 3, respectively.

TABLE 6

| | Control | Experimental | Experimental | Experimental | Experimental |
|---|---|---|---|---|---|
| Composition (wt %) | Group | Group 1 | Group 2 | Group 3 | Group 4 |
| Corn | 26.81 | 27.26 | 27.76 | 27.26 | 27.26 |
| Wheat | 10.00 | 10.00 | 10.00 | 10.00 | 10.00 |
| Cassava | 5.00 | 5.00 | 5.00 | 5.00 | 5.00 |
| Wheat bran | 9.42 | 9.42 | 9.42 | 9.42 | 9.42 |
| Corn germ meal | 4.00 | 4.00 | 4.00 | 4.00 | 4.00 |
| Soybean meal | 19.88 | 19.88 | 19.88 | 19.88 | 19.88 |
| Rapeseed meal | 3.00 | 3.00 | 3.00 | 3.00 | 3.00 |
| Dried distilled grains | 6.00 | 6.00 | 6.00 | 6.00 | 6.00 |
| Cookie by-product | 3.00 | 3.00 | 3.00 | 3.00 | 3.00 |
| Animal fat | 3.60 | 3.10 | 2.60 | 3.10 | 2.60 |
| Molasses | 6.00 | 6.00 | 6.00 | 6.00 | 6.00 |
| Liquid lysine (with HCl) | 0.28 | 0.28 | 0.28 | 0.28 | 0.28 |
| Liquid choline | 0.06 | 0.06 | 0.06 | 0.06 | 0.06 |
| Limestone | 1.24 | 1.24 | 1.24 | 1.24 | 1.24 |
| Di-calcium phosphate | 0.96 | 0.96 | 0.96 | 0.96 | 0.96 |
| Salt | 0.20 | 0.20 | 0.20 | 0.20 | 0.20 |
| Vitamin-mineral premix | 0.25 | 0.25 | 0.25 | 0.25 | 0.25 |
| Miscellaneous | 0.30 | 0.30 | 0.30 | 0.30 | 0.30 |
| Example 1 | — | 0.05 | 0.05 | — | — |
| Example 3 | — | — | — | 0.05 | 0.05 |

TABLE 7

| Chemical | Control | Experimental | Experimental | Experimental | Experimental |
|---|---|---|---|---|---|
| composition (wt %) | Group | Group 1 | Group 2 | Group 3 | Group 4 |
| Moisture | 11.84 | 11.83 | 11.89 | 11.83 | 11.89 |
| Crude protein | 17.49 | 17.54 | 17.58 | 17.54 | 17.58 |
| Crude fat | 7.08 | 6.62 | 5.67 | 6.62 | 5.67 |
| Crude fiber | 4.31 | 4.30 | 4.31 | 4.30 | 4.31 |
| Crude ash | 5.81 | 5.79 | 5.80 | 5.79 | 5.80 |
| Calcium | 0.80 | 0.80 | 0.80 | 0.80 | 0.80 |
| Total phosphorous | 0.55 | 0.55 | 0.55 | 0.55 | 0.55 |
| Available phosphorus | 0.30 | 0.30 | 0.30 | 0.30 | 0.30 |
| Total lysine | 0.98 | 0.99 | 0.99 | 0.99 | 0.99 |
| Net energy (kcal) | 2,320.00 | 2,295.00 | 2,270.00 | 2,295.00 | 2,270.00 |

The results of the present experiment were processed using the GLM procedures of the SAS (1985) statistical processing package, and Duncan's multiple range test was used to analyze final weight, daily weight gain (ADG), feed intake (ADFI), and feed conversion ratio.

TABLE 8

| | Control | Experimental | Experimental | Experimental | Experimental |
|---|---|---|---|---|---|
| Item | Group | Group 1 | Group 2 | Group 3 | Group 4 |
| Starting weight (kg) | 29.80 ± 0.98 | 29.90 ± 0.98 | 30.20 ± 1.12 | 29.85 ± 0.87 | 30.01 ± 0.91 |
| Final weight (kg) | 46.60 ± 1.72 | 48.40 ± 1.95 | 47.30 ± 2.31 | 48.05 ± 1.59 | 47.19 ± 2.05 |
| Feed intake (ADFI, kg) | 1.49 ± 0.02 | 1.56 ± 0.03 | 1.52 ± 0.02 | 1.57 ± 1.12 | 1.53 ± 0.53 |
| Daily weight gain | 0.600 ± 0.03 | 0.661 ± 0.05 | 0.611 ± 0.05 | 0.650 ± 0.07 | 0.614 ± 0.04 |
| (ADG, kg) | | | | | |
| Feed conversion ratio | 2.48 ± 0.11 | 2.36 ± 0.16 | 2.49 ± 0.16 | 2.42 ± 0.21 | 2.49 ± 0.62 |
| (FCR) | | | | | |

Referring to Table 8, there were no large significant differences in final weight amongst the treatment groups, and although there was no significant difference in average daily feed intake at a confidence level of 95%, a significant tendency for high final weight was exhibited by Experimental Group 1 at a confidence level of 90%.

In the case of daily weight gain, the value for Experimental Group 1 (with respect to the Control Group, 0.5% less animal fat, and addition of Example 1) was exhibited to be much higher than that of the Control Group. In addition, the daily weight gain of Experimental Group 2 (with respect to the Control Group, 1.0% less animal fat, and addition of Example 1) was also shown to be improved in comparison to that of the Control Group.

Through such results, it was concluded that when the feed additive of the present invention is added in place of a portion of the fat composition, even if the energy content in the feed is reduced by lowering the level of fat added into the feed to be 1.0% less than that of the Control Group and the cost of the feed is reduced, the digestion and utilization of consumed fat is improved such that there is no effect on daily weight gain.

Although there were no statistically significant differences in feed conversion rate amongst the treatment groups (P>0.05, P>0.1), it was shown that Experimental Group 1 was improved in comparison to the Control Group in terms of feed conversion rate. This result indicates that when an emulsifier is added, even if the level of fat added is 0.5% less than the Control Group, the digestion and utilization of consumed fat is improved such that there is an effect of improving feed conversion rate.

In conclusion, when 0.05% of the feed additive according to the present invention is added, productivity is shown to increase even if the energy content in the feed is reduced by lowering the level of fat added to 0.5% less than that of the Control Group (daily feed intake enhanced, P<0.1), and productivity is shown to be unaffected even if up to 1% of the added fat in the Control Group is replaced (P>0.1). Here, feed cost was shown to decrease.

### Experimental Example 4

### Test of Effect on Chicken Growth

Testing was carried out on a CP Indonesia farm. Testing was carried out by feeding grain to 14,000 chickens per coop. To observe the effect on growth of replacing a portion of the fat used in chicken feed with the feed additive of Examples 1 and 3, in Experimental Groups 1 and 3, 1% of palm oil was removed (replaced with corn) and 0.05% of the feed additive of Examples 1 and 3 respectively was added. The results obtained are displayed in Table 9.

TABLE 9

| | Control | Experimental | Experimental |
|---|---|---|---|
| Item | Group | Group 1 | Group 2 |
| Average market weight per | 1427.0 | 1436.0 | 1431.0 |
| chicken (kg) | | | |
| Production index | 344 | 354 | 350 |
| Feed conversion ratio (FCR) | 1.43 | 1.40 | 1.41 |

Referring to Table 9, in the case of the experimental groups in which the feed additive according to the present invention was consumed, a higher weight increase was observed even though the feed conversion ratio decreased. This result indicates that there is an improvement effect on the feed conversion ratio owing to improvements in digestion and utilization of fat in the feed, which are due to the feed additive contained in the feed.

An animal feed additive according to the present invention is used in animal feed and increases the utilization efficiency of fat that is present in the feed, thereby reducing the amount of fat required in the feed and enhancing livestock productivity. In particular, by increasing the content of a salt of lactic acid and palmitic acid ester or a salt of lactic acid and lauric acid ester in order to reduce the melting point of SSL, solubility in the bodies of animals is improved, and thus effective activity can be expected.

What is claimed is:

1. An animal feed additive comprising:
20 to 50 wt % of a salt of lactic acid and stearic acid ester; and
50 to 80 wt % of one kind of salt selected from the group consisting of a salt of lactic acid and palmitic acid ester, a salt of lactic acid and lauric acid ester, and mixtures thereof

2. The animal feed additive of claim 1, wherein the lactic acid is a monomer or a dimer.

3. The animal feed additive of claim 1, wherein the salt is a sodium salt, a calcium salt, or a potassium salt.

4. The animal feed additive of claim 1, wherein the animal feed additive is for enhancing the absorption ratio of fat present in the feed.

5. The animal feed additive of claim 4, wherein the feed is for raising pig, chicken, duck, quail, goose, pheasant, turkey, cattle, milk cow, horse, donkey, sheep, goat, dog, cat, rabbit, or farmed fish or shrimp.

6. An animal bile salt supplement comprising:
20 to 50 wt % of a salt of lactic acid and stearic acid ester; and
50 to 80 wt % of one kind of salt selected from the group consisting of a salt of lactic acid and palmitic acid ester, a salt of lactic acid and lauric acid ester, and mixtures thereof

7. The animal bile salt supplement of claim 6, wherein the lactic acid is a monomer of a dimer.

8. The animal bile salt supplement of claim 6, wherein the salt is a sodium salt, a calcium salt, or a potassium salt.

9. The animal bile salt supplement of claim 6, wherein the animal bile salt supplement is for enhancing the absorption ratio of fat present in the feed.

10. The animal bile salt supplement of claim 9, wherein the feed is for raising pig, chicken, duck, quail, goose, pheasant, turkey, cattle, milk cow, horse, donkey, sheep, goat, dog, cat, rabbit, or farmed fish or shrimp.

11. A method for reducing the content of fat required in feed by enhancing the absorption ratio of fat present in the feed, the method comprising feeding animals with the animal feed additive according to claim 1.

12. The method of claim 11, wherein the salt in the animal feed additive is in a non-dissociated state when fed to the animals.

13. The method of claim 11, wherein the animal feed additive is not in an emulsified state mixed with oil-based components and water-based components when fed to the animals.

14. The method of claim 11, wherein the livestock is pig, chicken, duck, quail, goose, pheasant, turkey, cattle, milk cow, horse, donkey, sheep, goat, dog, cat, rabbit, or farmed fish or shrimp.

15. A method for reducing the content of fat required in feed by enhancing the absorption ratio of fat present in the feed, the method comprising feeding animals with the animal bile salt supplement according to claim 6.

16. The method of claim 15, wherein the salt in the animal bile salt supplement is in a non-dissociated state when fed to the animals.

17. The method of claim 15, wherein the bile salt supplement is not in an emulsified state mixed with oil-based components and water-based components when fed to the animals.

18. The method of claim 15, wherein the livestock is pig, chicken, duck, quail, goose, pheasant, turkey, cattle, milk cow, horse, donkey, sheep, goat, dog, cat, rabbit, or farmed fish or shrimp.

19. A method for enhancing the productivity of livestock, wherein animals are fed with the animal feed additive of claim 1.

20. The method of claim 19, wherein the salt in the animal feed additive is in a non-dissociated state when fed to the animals.

21. The method of claim 19, wherein the animal feed additive is not in an emulsified state mixed with oil-based components and water-based components when fed to the animals.

22. The method of claim 19, wherein the livestock is pig, chicken, duck, quail, goose, pheasant, turkey, cattle, milk cow, horse, donkey, sheep, goat, dog, cat, rabbit, or farmed fish or shrimp.

23. A method for enhancing the productivity of livestock, wherein animals are fed with the animal bile salt supplement of claim 6.

24. The method of claim 23, wherein the salt in the animal bile salt supplement is in a non-dissociated state when fed to the animals.

25. The method of claim 23, wherein the animal bile salt supplement is not in an emulsified state mixed with oil-based components and water-based components when fed to the animals.

26. The method of claim 23, wherein the livestock is pig, chicken, duck, quail, goose, pheasant, turkey, cattle, milk cow, horse, donkey, sheep, goat, dog, cat, rabbit, or farmed fish or shrimp.

27. An animal feed composition comprising:
the animal feed additive of claim 1; and
a formulated feed.

28. The animal feed composition of claim 27, wherein the content of the animal feed additive is 0.01 to 5 wt % with respect to the total weight of the formulated feed.

29. The animal feed composition of claim 27, wherein the animal feed composition is for raising pig, chicken, duck, quail, goose, pheasant, turkey, cattle, milk cow, horse, donkey, sheep, goat, dog, cat, rabbit, or farmed fish or shrimp.
